(19) United States
(12) Patent Application Publication
(10) Pub. No.: US 2023/0389995 A1
(43) Pub. Date: Dec. 7, 2023

(54) DISTAL TIP TRACKING AND MAPPING

(71) Applicant: BOSTON SCIENTIFIC SCIMED, INC., Maple Grove, MN (US)

(72) Inventors: Kian S. LIM, Shrewsbury, MA (US); Longquan CHEN, Lexington, MA (US)

(21) Appl. No.: 18/297,728

(22) Filed: Apr. 10, 2023

Related U.S. Application Data
(60) Provisional application No. 63/365,911, filed on Jun. 6, 2022.

(51) Int. Cl. A61B 34/20; A61B 5/06

(52) U.S. Cl. CPC A61B 34/20; A61B 5/06; A61B 2034/2046

(57) ABSTRACT

A system is for tracking includes a longitudinal element extending longitudinally from a distal tip inserted into a body to a proximal end which remains outside of the body. The element includes an IMU sensor at the tip and a series of stripes extending along the element. Data from the IMU sensor determines an angular orientation of the tip. The system also includes an exterior member positioned outside of the body. The member includes a channel extending longitudinally therethrough and a linear encoder detecting each of the stripes of the element as the element is slid through the member so that data from the encoder determines a linear displacement of the element relative to the member at a point of entry. 3D location of the tip calculated based on the determined angular orientation and the linear displacement determined at each detected stripe.

### PRIORITY CLAIM

The present disclosure claims priority to U.S. Provisional patent application Ser. No. 63/365,911 filed Jun. 6, 2022; the disclosure of which is incorporated herewith by reference.

### FIELD

The present disclosure relates to endoscopic devices and, in particular, relates to endoscopic device configured such that a precise location of a distal tip thereof may be tracked,

### BACKGROUND

Many endoscopic procedures such as, for example, gastrotomy, ERCP (Endoscopic Retrograde Cholangiopancreatography), and lithotripsy, require an endoscope to be navigated through a portion of a patient's body to provide treatment to target tissue within the body. In some endoscopic procedures, fluoroscopy may be used to image an internal structure and function of an anatomy or organ of the patient to be treated. Fluoroscopy, however, uses X-rays to obtain a continuous image, exposing the patient to radiation. In addition, fluoroscopy is unable to provide a three-dimensional location of a distal tip of an endoscope within the body.

### SUMMARY

The present disclosure relates to a system for tracking a distal tip of a longitudinal element inserted into a patient body. The system includes a longitudinal element extending longitudinally from a distal tip configured for insertion into a target area within an anatomy of the patient body to a proximal end configured to remain outside of the patient body. The longitudinal element includes an IMU sensor at the distal tip and a series of stripes extending along the longitudinal element. Data from the IMU sensor is determining an angular orientation of the distal tip of the longitudinal element. In addition, the system includes an exterior member configured to be positioned outside of the patient body at a point of entry into the target area of the patient body. The exterior member includes a channel extending longitudinally therethrough and a linear encoder configured to detect each of the stripes of the longitudinal element as the longitudinal element is slid through the exterior member so that data from the linear encoder determines a linear displacement of the longitudinal element relative to the exterior member at the point of entry. A three-dimensional location of the distal tip is calculated based on the determined angular orientation and the linear displacement determined at each detected stripe.

In an embodiment, the linear encoder includes a first optical fiber and a second optical fiber, the first optical fiber and a second optical fiber detecting a first linear displacement and a second linear displacement, respectively, of the longitudinal element at the point of entry to determine a linear direction of movement of the longitudinal element relative to the exterior member.

In an embodiment, each of the stripes extend about a periphery of the longitudinal element and is separated from an adjacent one of the stripes via a predetermined distance.

In an embodiment, the stripes are equidistantly spaced from one another.

In an embodiment, the longitudinal element is one of an endoscope and an endotracheal tubing.

In an embodiment, the angular orientation of the distal tip is determined via a sensor fusing technology.

In an embodiment, the location of the distal tip is determined by performing a motion decomposition of the angular orientation and calculating an integral of the decomposed motion.

In an embodiment, the location of the distal tip for each detected stripe is determined to track a three-dimensional motion of the distal tip.

In an embodiment, the three-dimensional motion of the distal tip is displayed on a display.

In an embodiment, the linear encoder includes a camera sensing changes in image intensity to detect each of the stripes.

In addition, the present disclosure relates to a system for tracking a distal tip of a longitudinal element inserted into a patient body. The system includes a longitudinal element extending longitudinally from a distal tip configured for insertion into a target area within an anatomy of the patient body to a proximal end configured to remain outside of the patient body. The longitudinal element includes an IMU sensor at the distal tip and a series of stripes extending along the longitudinal element. Each of the stripes is separated from an adjacent one of the stripes via a predetermined distance. The system also includes an exterior member configured to be positioned outside of the patient body at a point of entry into the target area of the patient body. The exterior member includes a channel extending longitudinally therethrough and a linear encoder configured to detect each of the stripes of the longitudinal element as the longitudinal element is slid through the exterior member. The system further includes a processor configured to receive data from the IMU sensor and the linear encoder to determine an angular orientation of the distal tip of the longitudinal element from data received from the IMU sensor and a linear displacement of the longitudinal element relative to the exterior member from data received from the linear encoder, the processor configured to determine a three-dimensional location of the distal tip for each detected stripe.

In an embodiment, the linear encoder includes a first optical fiber and a second optical fiber, the first optical fiber and a second optical fiber detecting a first linear displacement and a second linear displacement, respectively, of the longitudinal element at the point of entry to determine a linear direction of movement of the longitudinal element relative to the exterior member.

In an embodiment, the processor determines the angular orientation of the distal tip via a sensor fusing technology.

In an embodiment, the processor determines the location of the distal tip by performing a motion decomposition of the angular orientation and calculating an integral of the decomposed motion.

In an embodiment, the system further incudes a display configured to display a tracked three-dimensional motion of the distal tip.

Also, the present disclosure relates to a method for tracking a distal tip of a longitudinal element. The method includes positioning an exterior member including a linear encoded mounted therein outside a patient body at a point of entry into the patient body; sliding a longitudinal element through the exterior member and into a target area of an anatomy of the patient body, the longitudinal element extending from a distal tip configured for insertion into the target area to a proximal end configured to remain outside of the patient body, the longitudinal element including an IMU sensor at the distal tip and a series of stripes extending along the longitudinal element, each of the stripes separated from an adjacent one of the stripes via a predetermined distance; determining an angular orientation of the distal tip of the longitudinal element from data received from the IMU sensor and a linear displacement of the longitudinal element relative to the exterior member from data received from a linear encoder; and determining a three-dimensional location of the distal tip for each detected stripe.

In an embodiment, the linear displacement of the longitudinal element includes a distance and direction of linear displacement.

In an embodiment, the angular orientation of the distal tip is determined via a sensor fusing technology.

In an embodiment, the three-dimensional location of the distal tip is determined by performing a motion decomposition of the angular orientation and calculating an integral of the decomposed motion.

In an embodiment, the method further includes tracking a three-dimensional motion of the distal tip based on the three-dimensional location of the distal tip at each detected stripe.

### BRIEF DESCRIPTION

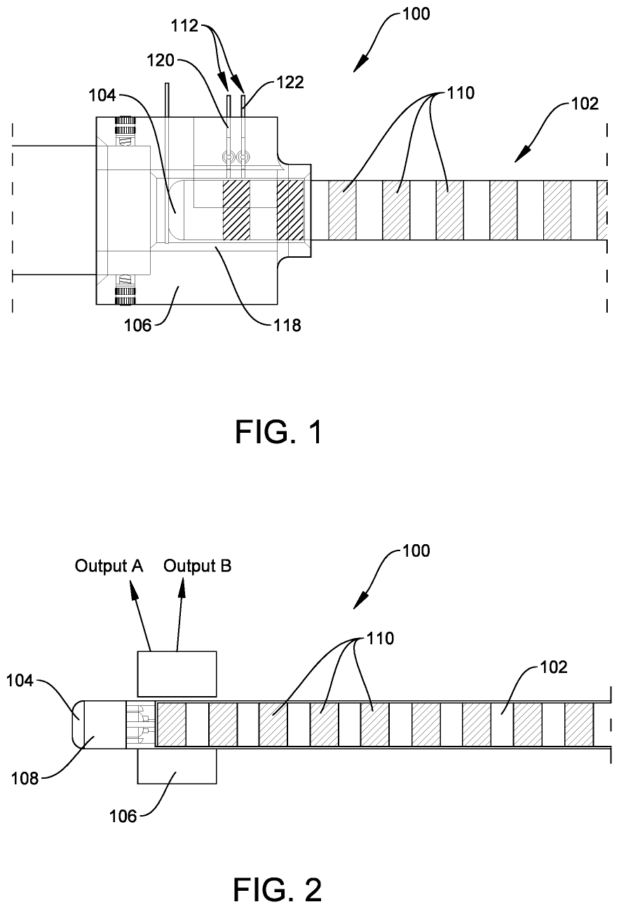

FIG. 1 shows a longitudinal side view of a distal portion of a system for tracking a distal tip of a longitudinal element configured for insertion into a body, according to an exemplary embodiment of the present disclosure;

FIG. 2 shows another longitudinal side view of the distal portion of the system according to FIG. 1;

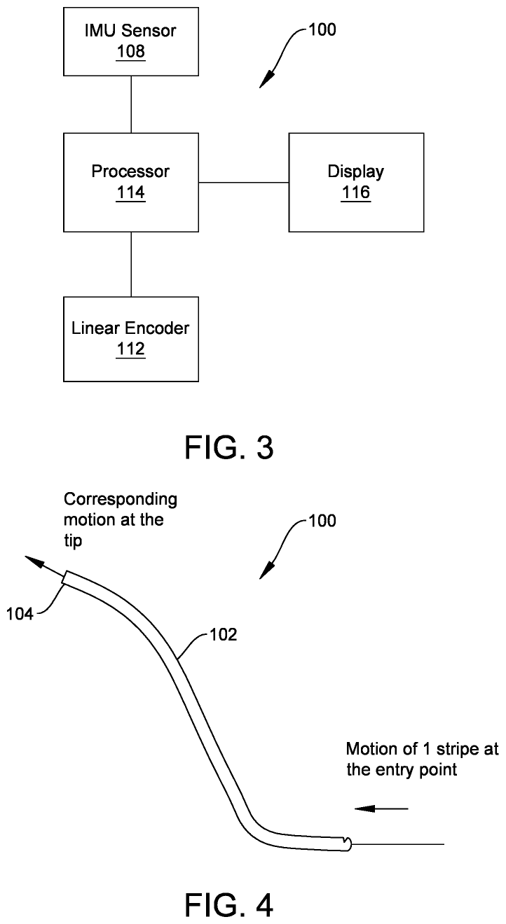

FIG. 3 shows a schematic diagram of the system according to FIG. 1;

FIG. 4 shows a schematic drawing illustrating a motion of the longitudinal element of the system according to FIG. 1;

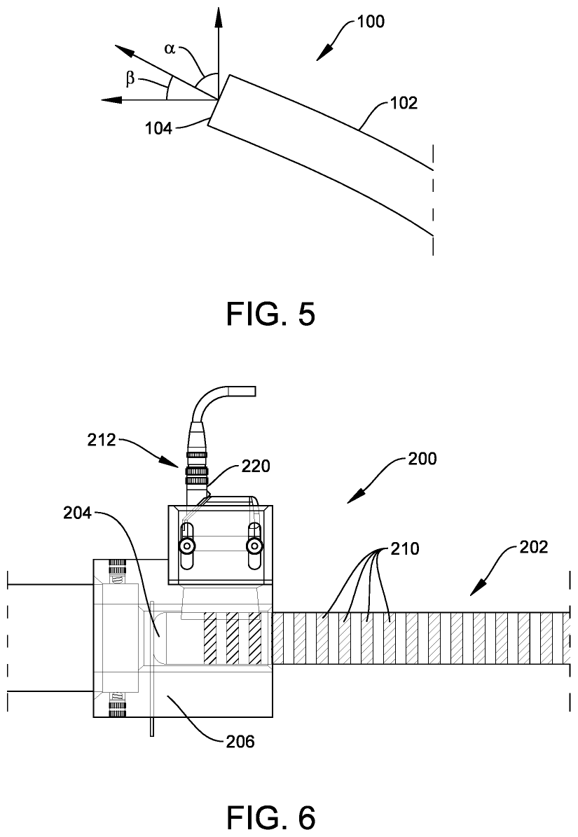

FIG. 5 shows a schematic drawing illustrating an angular motion at a distal tip of the longitudinal element of the system according to FIG. 1; and

FIG. 6 shows a longitudinal side view of a distal portion of a system for tracking a distal tip of a longitudinal element configured for insertion into a body, according to another exemplary embodiment of the present disclosure.

### DETAILED DESCRIPTION

The present disclosure may be further understood with reference to the following description and the appended drawings, wherein like elements are referred to with the same reference numerals. The present disclosure relates to a tracking system and, in particular, relates to a tracking system in which three-dimensional movement of a distal tip of an endoscope, or other longitudinal element, may be tracked. Exemplary embodiments of the present disclosure comprise a longitudinal element (e.g., endoscope) configured to be inserted into a living body, the longitudinal element including an IMU (Inertial Measurement Units) sensor at a distal tip thereof and a series of stripes printed along the longitudinal element. The stripes are separated from one another along the longitudinal element by a predetermined distance. The longitudinal element is slidable through a structure including linear encoder components which measure linear motion (e.g., distance) of the longitudinal element as it is slid therethrough.

The IMU sensor and the linear encoding components are in communication with a processor (e.g., wirelessly or via a wired electrical coupling), which determines a precise location of the distal tip of the longitudinal element based on angular motion (e.g., orientation) of the distal tip determined via data from the IMU sensor and the linear motion detected via the linear components. The processor determines the precise location of the distal tip for each stripe detected via the linear encoder components so that the three-dimensional movement of the distal tip may be tracked. It will be understood by those of skill in the art that the distal tip tracking provided by the exemplary embodiments aids in navigation of the longitudinal element through the body of the patient to the target area, reduces exposure to radiation from, for example, fluoroscopy and, when used in conjunction with, for example, an endoscopic vision system, may provide anatomical mapping. It will also be understood by those of skill in the art that terms proximal and distal, as used herein, are intended to refer to a direction toward and away from, respectively, a user of the device.

A tracking system 100, as shown in FIGS. 1-5, determines a precise location of a distal tip 104 of a longitudinal element 102 such as, for example, an endoscope, which is inserted into a living body to a target area within an anatomical region or organ to be treated. The tracking system 100 comprises the longitudinal element 102 configured to be inserted into the body and an exterior member 106 configured to be positioned outside of the body, at a point of entry into the body—e.g., at a body orifice through which the longitudinal element 102 is to be inserted into the body.

The longitudinal element 102 is configured to be slid through the exterior member 106 and includes an IMU sensor 108 at the distal tip 104 of the longitudinal element 102 and a series of stripes 110 along the longitudinal element 102, each of the stripes 110 separated from one another by a predetermined distance. The exterior member 106 includes a linear encoder 112, which detect the stripes 110 as the longitudinal element 102 is slid through the exterior member, to determine a linear motion of the distal tip 104. Both the IMU sensor 108 and the linear encoder 112 are in communication with a processor 114 so that the processor receives data from the IMU sensor 108 and the linear encoder 112 to determine an angular orientation of the distal tip 104 and linear motion of the longitudinal element 102 relative to the point of entry.

Based on the determined angular orientation of the distal tip 104 and the linear displacement of the longitudinal element 102, the processor 114 determines a three-dimensional location of the distal tip 104. The processor 114 determines the location of the distal tip 104 for each stripe 110 detected via the linear encoder 112. According to an exemplary embodiment, the tracking system 100 further comprises a display 116 on which the continuous three-dimensional tracking of the distal tip 104 may be shown.

As shown in FIGS. 1-2, the longitudinal element 102 extends longitudinally from the distal tip 104 configured to be inserted into the body to a proximal end, which remains outside the body accessible to a user (e.g., physician) of the system 100. In one embodiment, the longitudinal element 102 is any of a variety of endoscopes including, for example, a ureteroscope. It will be understood by those of skill in the art, however, that the longitudinal element 102 may be any of a variety of longitudinal elements configured to be inserted into a body to treat target areas within the body. In one example, the longitudinal element 102 includes an endotracheal tubing.

The distal tip 104 of the longitudinal element 102 includes the IMU sensor 108. The IMU sensor 108 may be embedded within the distal tip 104, as shown in FIG. 2. The IMU sensor 108 is an electronic device used to calculate and report an exact force on an element, a rate of change of an angular orientation of the element, as well as a direction of movement of the element. The IMU sensor 108 is a blend of three sensors including a gyroscope, a magnetometer, and an accelerometer to determine motion detection, and orientation about multiple axes as well as forces exerted on the body. The IMU sensor 108 is thus also capable of performing motion tracking. However, IMU-based tracking may not be reliable for long-term, tracking as it calculates positions by double integration of measured accelerations. As calculation errors with double integration grow exponentially with time, calculated positions drift over time, resulting in what is known as “IMU localization drift.”

As will be described in greater detail below, the longitudinal element 102 includes the series of stripes 110, which are detectable via the linear encoder 112, to eliminate the IMU localization drift. According to an exemplary embodiment, the series of stripes 110 may be printed, laser cut or etched along the longitudinal element 102. Each stripe 110, in an exemplary embodiment extends about a circumference of the longitudinal element 102 and the stripes 110 are separated from one another along the longitudinal element 102 by a predetermined distance known to the system.

In one embodiment, the stripes 110 are equidistantly separated from one another. The stripes 110 may have any of a variety of colors and/or configurations so long as each of the stripes 110 is detectable via the linear encoder 112. In one embodiment, the stripes 110 may be black in color. The stripes 110 should extend along at least a portion of a length of the longitudinal element 102 configured to be inserted into the patient body.

The exterior member 106 includes the linear encoder 112 mounted therein and is configured to be positioned at a point of entry into the patient body. As described above, this may, in one example, include a location adjacent to a body orifice through which the longitudinal element 102 is to be inserted. According to an exemplary embodiment, the exterior member 106 may be positioned against or immediately adjacent to the point of entry of the device into the body and, in one embodiment, is configured to be attached to the point of entry.

According to an exemplary embodiment, the exterior member 106 has a substantially tubular configuration including a channel 118 extending therethrough so that the longitudinal element 102 is longitudinally slidable through the channel 118. As the longitudinal element 102 slides through the channel 118, the linear encoder 112 detects each of the stripes 110 to determine, among other things, the length of the portion of the device that has been inserted into the body.

In one embodiment, the linear encoder 112 includes a pair of optical fibers—a first optical fiber 120 and a second optical fiber 122. Each of the first and second optical fibers 120, 122 is able to detect the stripes 110 and/or the non-striped portions extending therebetween to determine a linear displacement of the longitudinal element 102 into and/or out of the body. In other words, a distance over which the longitudinal element 102 has moved distally and/or proximally through the exterior member 106 is detected. In this embodiment, the linear encoder 112 includes the first and second optical fibers 120, 122 so that each of the optical fibers 120, 122 outputs a displacement—Output A, Output B, respectively. Each of outputs A, B represents a linear displacement of the longitudinal element 102 relative to the point of entry into the body. The two outputs A, B, allow the processor 114 to determine whether the distal tip 104 is moving forward (i.e., distally) or backward (i.e., proximally).

As shown in FIG. 3, the IMU sensor 108 and the linear encoder 112 are in communication with the processor 114. The IMU sensor 108 and the linear encoder 112 may be wirelessly and/or electrically coupled to the processor 114 so that the processor 114 may receive data from each of the IMU sensor 108 and the linear encoder 112. The processor 114 of this embodiment includes a computing device configured to execute computer-executable instructions for operations that provide functionalities to the system 100.

For example, as will be described in further detail below, the processor 114 executes instructions for determining orientation information of the distal tip 104, determining linear motion of the longitudinal element 102, and determining, based on the orientation information and the linear motion, a three-dimensional location of the distal tip 104. The processor 114 of this embodiment is connected to the display 116, which shows/displays results of the tracking system 100—e.g., three-dimensional motion tracking of the distal tip 104. The processor 114 and the display 116 in an exemplary embodiment are components of a computing device in communication with the IMU sensor 108 and the linear encoder 112 of the system 100.

In another embodiment, these elements are modular components connected to the linear encoder 112 of the system 100. It will be understood by those of skill in the art that a system 100 according to an embodiment may include any of the components described above in any combination permitting the three-dimensional tracking of the distal tip 104.

As shown in FIG. 4, the linear encoder 112 determines a one-dimensional displacement of the longitudinal element 102 at the point of entry—i.e., the location of the exterior member 106. This linear displacement, however, is then translated to an angular motion of the distal tip 104, as shown in FIGS. 4-5, which is determined via data from the IMU sensor 108. The linear motion and the angular motion together determine the three-dimensional location of the distal tip 104 as would be understood by those skilled in the art. In an exemplary embodiment, the angular motion (e.g., orientation) of the distal tip 104 is determined with each detected change in the one-dimensional displacement of the longitudinal element 102 occurs.

In particular, when the linear encoder 112 detects the passing of a stripe 110 and determines that a linear displacement has occurred, the processor 114 calculates orientation information using sensor fusing technology. In particular, since gyroscopes often have a drifting problem, the accelerometer sensor of an exemplary embodiment is used to correct the gyroscope sensor to provide accurate orientation information using sensor fusing technology.

According to an exemplary embodiment, the processor 114 performs motion decomposition based on the orientation data received from the IMU sensor 108. The processor 114 then determines the tip position based on the decomposed motion. In an embodiment, the tip position is determined by calculating an integral of the decomposed motion. Since tip location is determined as the passing of each stripe 110 is detected, the processor 114 is able to track three-dimensional motion of the distal tip 104. As described above, the first and second optical fibers 120, 122 are also able to track a longitudinal direction of movement of the longitudinal element 102 relative to the exterior member 106. The tracked motion may be displayed on the display 116. Since the tip position is determined in this embodiment via only one integral operation, there are no drifting problem as may occur with tracking via an IMU sensor alone. Thus, the system 100 provides an accurate location of the distal tip 104.

According to an exemplary method for tracking the distal tip 104 of the longitudinal element 102 of the system 100, the exterior member 106 is positioned outside of the body, at the point of entry into the body of the patient. In one embodiment, this may be the opening of a body orifice into a target area of an anatomy to be treated. Then, as longitudinal element 102 is slid through the channel 118 of the exterior member 106, the linear encoder 112 of the exterior member 106 detects each stripe 110 that passes through the exterior member 106.

As each stripe 110 is detected, angular motion of the distal tip 104 is also determined via data received from the IMU sensor 108. The processor 114 then performs motion decomposition based on the angular motion/orientation data. The location of the distal tip 104 is calculated by taking the integral of this decomposed motion. As discussed above, the location of the distal tip 104 is determined for each stripe 110 detected as the longitudinal element 102 is slid though the exterior member 106. As also discussed above, the first and second optical fibers 120, 122 of the linear encoder 112 are able to determine a direction of movement of the longitudinal element 102—i.e., whether the longitudinal element is being inserted into the body via a distal motion or withdrawn from the body via a proximal motion. As a location of the distal tip 104 is continuously determined, three-dimensional movement of the distal tip 104 is tracked based on the location of the distal tip 104 determined for each stripe 110 and displayed on the display 116.

As shown in FIG. 6, a tracking system 200 according to another exemplary embodiment of the present disclosure may be substantially similar to the system 100, comprising a longitudinal element 202 slidable through an exterior member 206. Similarly to the system 100, the longitudinal element 202 is configured for insertion into a living body to a target area therewithin to be treated and may include, for example, a flexible endoscope, endotracheal tubing, etc. The exterior member 206 is configured to be positioned outside of the body, at a point of entry into the body—e.g., at an opening of a body orifice through which the longitudinal element 102 is to be inserted.

Similarly to the longitudinal element 102, the longitudinal element 202 includes an IMU sensor (not shown) at a distal tip 204 of the longitudinal element 202, along with a series of stripes 210 therealong. The stripes 210 extend at least partially circumferentially around and are separated from one another along the longitudinal element 202. The stripes 210 are separated from one another via predetermined distance which may be a known constant separation distance or a distance that varies in any known pattern so long as the system 200 can determine based on the passing of each stripe 210 through the exterior member 206 a distance represented between the stripe 210 and a stripe 210 that had previously passed through the exterior member 206. The exterior member 206 also includes a linear encoder 212, which detects the stripes 210 as the longitudinal element 202 is slid through the exterior member 206, to determine a linear motion of the distal tip 204.

The system 200 is substantially similar to the system 100 except that the linear encoder 212 in this embodiment includes a camera 220 for detecting the stripes 210 rather than optical fibers, as described above with respect to the system 100. The camera 220 detects changes in image intensity to identify the stripes 210 along the longitudinal element 202 as they pass through the exterior member 206. Similarly to the system 200, the IMU sensor and the linear encoder (e.g., camera 220) are in communication with a processor, which receives data IMU sensor and the linear encoder 212 to determine an angular orientation of the distal tip 204 and a linear motion of the longitudinal element 202 relative to the point of entry, respectively.

Based on the determined angular orientation of the distal tip 204 and the linear displacement of the longitudinal element 202 and prior three-dimensional locations of the distal tip 204, the processor determines a three-dimensional location of the distal tip 204. The processor determines a location of the distal tip 204 with passing through the exterior member 206 of each stripe 210 detected via the linear encoder 212 to provide a three-dimensional tracking of the distal tip 204, which may be displayed on a display.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. A system for tracking a distal tip of a longitudinal element inserted into a patient body, comprising:
a longitudinal element extending longitudinally from a distal tip configured for insertion into a target area within an anatomy of the patient body to a proximal end configured to remain outside of the patient body, the longitudinal element including an IMU sensor at the distal tip and a series of stripes extending along the longitudinal element, data from the IMU sensor determining an angular orientation of the distal tip of the longitudinal element; and
an exterior member configured to be positioned outside of the patient body at a point of entry into the target area of the patient body, the exterior member including a channel extending longitudinally therethrough and a linear encoder configured to detect each of the stripes of the longitudinal element as the longitudinal element is slid through the exterior member so that data from the linear encoder determines a linear displacement of the longitudinal element relative to the exterior member at the point of entry, a three-dimensional location of the distal tip calculated based on the determined angular orientation and the linear displacement determined at each detected stripe.

17. The system of claim 16, wherein the linear encoder includes a first optical fiber and a second optical fiber, the first optical fiber and a second optical fiber detecting a first linear displacement and a second linear displacement, respectively, of the longitudinal element at the point of entry to determine a linear direction of movement of the longitudinal element relative to the exterior member.

18. The system of claim 16, wherein each of the stripes extend about a periphery of the longitudinal element and is separated from an adjacent one of the stripes via a predetermined distance.

19. The system of claim 18, wherein the stripes are equidistantly spaced from one another.

20. The system of claim 16, wherein the longitudinal element is one of an endoscope and an endotracheal tubing.

21. The system of claim 16, wherein the angular orientation of the distal tip is determined via a sensor fusing technology.

22. The system of claim 16, wherein the location of the distal tip is determined by performing a motion decomposition of the angular orientation and calculating an integral of the decomposed motion.

23. The system of claim 16, wherein the location of the distal tip for each detected stripe is determined to track a three-dimensional motion of the distal tip.

24. The system of claim 23, wherein the three-dimensional motion of the distal tip is displayed on a display.

25. The system of claim 16, wherein the linear encoder includes a camera sensing changes in image intensity to detect each of the stripes.

26. A system for tracking a distal tip of a longitudinal element inserted into a patient body, comprising:
a longitudinal element extending longitudinally from a distal tip configured for insertion into a target area within an anatomy of the patient body to a proximal end configured to remain outside of the patient body, the longitudinal element including an IMU sensor at the distal tip and a series of stripes extending along the longitudinal element, each of the stripes separated from an adjacent one of the stripes via a predetermined distance; and
an exterior member configured to be positioned outside of the patient body at a point of entry into the target area of the patient body, the exterior member including a channel extending longitudinally therethrough and a linear encoder configured to detect each of the stripes of the longitudinal element as the longitudinal element is slid through the exterior member; and
a processor configured to receive data from the IMU sensor and the linear encoder to determine an angular orientation of the distal tip of the longitudinal element from data received from the IMU sensor and a linear displacement of the longitudinal element relative to the exterior member from data received from the linear encoder, the processor configured to determine a three-dimensional location of the distal tip for each detected stripe.

27. The system of claim 26, wherein the linear encoder includes a first optical fiber and a second optical fiber, the first optical fiber and a second optical fiber detecting a first linear displacement and a second linear displacement, respectively, of the longitudinal element at the point of entry to determine a linear direction of movement of the longitudinal element relative to the exterior member.

28. The system of claim 26, wherein the processor determines the angular orientation of the distal tip via a sensor fusing technology.

29. The system of claim 26, wherein the processor determines the location of the distal tip by performing a motion decomposition of the angular orientation and calculating an integral of the decomposed motion.

30. The system of claim 26, further comprising a display configured to display a tracked three-dimensional motion of the distal tip.

31. A method for tracking a distal tip of a longitudinal element, comprising:
positioning an exterior member including a linear encoded mounted therein outside a patient body at a point of entry into the patient body;
sliding a longitudinal element through the exterior member and into a target area of an anatomy of the patient body, the longitudinal element extending from a distal tip configured for insertion into the target area to a proximal end configured to remain outside of the patient body, the longitudinal element including an IMU sensor at the distal tip and a series of stripes extending along the longitudinal element, each of the stripes separated from an adjacent one of the stripes via a predetermined distance;
determining an angular orientation of the distal tip of the longitudinal element from data received from the IMU sensor and a linear displacement of the longitudinal element relative to the exterior member from data received from a linear encoder; and
determining a three-dimensional location of the distal tip for each detected stripe.

32. The method of claim 31, wherein the linear displacement of the longitudinal element includes a distance and direction of linear displacement.

33. The method of claim 31, wherein the angular orientation of the distal tip is determined via a sensor fusing technology.

34. The method of claim 31, wherein the three-dimensional location of the distal tip is determined by performing a motion decomposition of the angular orientation and calculating an integral of the decomposed motion.

35. The method of claim 31, further comprising tracking a three-dimensional motion of the distal tip based on the three-dimensional location of the distal tip at each detected stripe.
